(19) United States
(12) Patent Application Publication
(10) Pub. No.: US 2004/0073259 A1
(43) Pub. Date: Apr. 15, 2004

(54) Treatment of patients infected by disease causing organisms or biological warfare agents using hyperthermia

(76) Inventor: Martin Munzer, Parkland, FL (US)

Correspondence Address: DUANE A. STEWART III, ESQ., BUCHANAN INGERSOLL PC, ONE OXFORD CENTRE, 20th FLOOR, 301 GRANT STREET, PITTSBURGH, PA, 15219, US

(21) Appl. No.: 10/649,544

(22) Filed: Aug. 26, 2003

Related U.S. Application Data
(60) Provisional application No. 60/406,478, filed on Aug. 28, 2002.

(51) Int. Cl. A61N 1/00

(52) U.S. Cl. 607/1

(57) ABSTRACT

The invention provides a method of treating a patient infected with any contaminant and/or with any biological/chemical warfare agent wherein the contaminant is other than a human hepatitis C virus, an HIV virus, or the human herpes virus comprising raising the core temperature of the patient and then returning the core temperature of the patient to normal at least one time. Preferably, the patient is an animal infected with an equine herpes virus, a contaminant that has caused an equine encephalitis infection, or a West Nile Virus.

### BENEFIT OF PRIOR PROVISIONAL APPLICATION

This utility patent application claims the benefit of co-pending U.S. Provisional Patent Application Serial No. 60/406,478, filed Aug. 28, 2002, entitled “Treatment of Patients Infected by Disease Causing Organisms or Biological Warfare Agents Using Hyperthermia,” having the same named applicant as inventor, namely, Martin Munzer. The entire contents of U.S. Provisional Patent Application Serial No. 60/406,478 are incorporated by reference into this utility patent application.

### BACKGROUND OF THE INVENTION

1. Field of the Invention

This invention relates to hyperthermic treatment of disease conditions in patients.

2. Description of the Background Art

Fever is induced by the immune system as one mechanism by which a mammal fights disease. A number of pathogens, including some bacteria, some cancers, and some viruses are adversely affected by heat. In addition, certain processes that normally fight disease, such as tumor necrosis factor A, seem to be stimulated by hyperthermia.

Hippocrates first described hyperthermia treatments, around 480 BC, which used hot sand baths for patients with skin tumors. In 1927 a Nobel prize was awarded to a doctor, Warner Jauregg who used malaria-induced fever to treat syphilis. However, by the mid-1930s the medical community began to recognize the potential hazards of hyperthermic therapy. Among the adverse effects of hyperthermia are increases in cardiac output, increases in oxygen consumption, changes in serum enzymes, drops in phosphate, calcium, and magnesium levels, and heart, liver and brain damage.

Hyperthermia has been induced using hot baths, bacterial inoculation, hot wax, hot air systems, heated water blankets, etc.

Two shortcomings of prior art systems and methods have been the lack of strict control over the duration and temperature exposure of the patient and the insufficient monitoring and control of numerous physiological parameters.

It is common for members of the animal kingdom to become infected by a variety of naturally occurring pathogens. Many of these pathogens have evolved to elude or evade the immune system by way of many complex mechanisms. The result of such evasiveness makes a successful therapeutic treatment difficult and at times impossible. A key to defeating some of these pathogens is to force them into an active proliferation at a time when conditions are not conducive to their survival. Improved methods for treating patients are being sought that can boost the natural immune response and/or disrupt the protective mechanism of the pathogens.

Biological warfare agents are designed to quickly spread disease and incapacitate and kill those exposed. Methods are sought that will improve the survival rates of patients who do not respond to conventional treatments or whose disease state has progressed beyond the ability of conventional treatments.

Scientific frontiers have reached the stage of knowledge that allows for any organism to be genetically modified. The splicing of a human interleukin gene into the genome of a virus, for example, renders the modified virus invisible to the immune system allowing the virus to destroy its victim without resistance. Methods are sought that will quickly allow for the treatment of patients infected with genetically modified biological warfare agents without the need of having the knowledge of which pathogen has been used in a biological attack.

### SUMMARY OF THE INVENTION

This invention provides a method for treating a patient infected with an organism or other heat-sensitive contaminant comprising raising the core temperature of the patient and then returning the core temperature of the patient to normal at least one time. The core temperature is raised to a temperature range, a duration, and a number of times sufficient to reduce or eliminate the patient's pathogenic load of an organism or contaminant.

It is to be understood that both the foregoing general description and the following detailed description are exemplary and explanatory and are intended to provide further explanation of the invention as claimed.

Although use of the invention is contemplated for a wide variety of organisms and other contaminants, notable uses include elimination of biological warfare agents, chemical warfare agents, genetically modified biological warfare agents, mutated cells, prions, bacteria, spirochetes, viruses, yeasts, parasites, fungi, equine herpes virus, West Nile Virus, and an organism that causes equine encephalitis. The invention is also contemplated for use during treatment of a patient with primary and secondary infections, where the secondary infection-causing contaminants may be, among other things, biological warfare agents, chemical warfare agents, genetically modified biological warfare agents, mutated cells, prions, bacteria, spirchetes, viruses, yeasts, parasites, fungi, equine herpes virus, West Nile virus, and an organism that causes equine encephalitis.

### BRIEF DESCRIPTION OF THE DRAWINGS

FIG. 1 is a simplified perspective view of an apparatus used to practice the invention.

FIG. 2 is a mechanical diagram showing cannulation sites on a human adult.

FIG. 3 is a simplified diagram of the system illustrated in FIG. 2.

FIG. 4 is a cross-section of a temperature sensor.

FIG. 5 is a cross-section of a temperature catheter having a temperature sensor positioned at the urinary sphincter muscle with the aid of an inflatable cuff that engages the bladder wall.

FIG. 6 is a cross-section of temperature catheter having two temperature sensors, one of which is positioned at the urinary sphincter muscle with the aid of an inflatable cuff that engages the bladder wall and the second of which is positioned in the urine pool.

### DETAILED DESCRIPTION OF THE INVENTION

The invention provides a method for treating a patient infected with an organism comprising raising the core temperature of the patient and then returning the core temperature of the patient to normal at least one time. As used herein the terms “patient” or “patients” mean members of the animal kingdom, including such as for example but not limited to human beings, non-human primates, horses, cats, dogs, and other members of the animal kingdom. The core temperature is raised to a temperature range, a duration, and a number of times sufficient to reduce or eliminate the patient's pathogenic load of an organism. “Treating” in this application means raising the core temperature to a temperature range, a duration, and a number to times sufficient to reduce or eliminate the patient's pathogenic load of an organism. Raising the core temperature upregulates an organism. At the high temperatures encountered with hyperthermia, and the up-regulation of the immune system (granulocytosis etc.) an organism is dispersed into a hostile environment where antibody and cell mediated responses can occur. Pharmaceutical administration prior to, during, or after this time frame could be devastating to the various organisms activated.

“Returning the core temperature of the patient to normal” includes allowing the patient to cool through ambient heat loss and actively cooling the patient. In the examples described below, the patient is cooled by ambient heat loss and active cooling to a temperature of about 39° C. The patient is released from the treatment area and the patient's temperature gradually returns to normal (about 37° C. for a human patient) over a period of a few days. It would be appreciated by one skilled in the art that the core temperatures of non-human patients may vary by species or individual, and that accepted core temperatures are available in the literature of the art. In one embodiment, the core temperature of the patient is raised and returned to normal one time. In another embodiment, the core temperature of the patient is raised and returned to normal two or more times. In one embodiment, the core temperature is raised by circulating the patient's blood from the patient, through an extracorporeal blood flow circuit, and back to the patient, wherein the blood returned to the patient has been heated within the blood flow circuit to an elevated temperature range. The patient's blood can be circulated from the patient through a blood vessel and returned to the patient through a blood vessel. In one embodiment, the patient's blood is circulated from the patient through a vein and returned to the patient through a vein. In another embodiment, the patient's blood is circulated from the patient through an artery and returned to the patient through a vein. In another embodiment, the core temperature is raised by inserting a heating element into the patient and the heating element heats the patient's blood. The heating element can be inserted into a blood vessel of the patient.

The heating element can be inserted into a central vessel, i.e., aorta or vena cava, where it can heat the blood passing by and eventually heating the blood to such a degree that the net temperature gain exceeds the losses due to the normal (physiologic) cooling mechanisms. Over time the body temperature can be raised to a predetermined point and maintained for a predetermined time. The heating element can be housed within a sheath or catheter at one or multiple positions along its length. The sheath or catheter can contain wires, conduits, fiberoptic, or other materials to supply power to the heating element. External to the body there could be a plug to connect the sheath or catheter to the control system. The sheath or catheter can be treated to give it antithrombogenic properties. This treatment can be chemical or a high energy corona or plasma discharge in the presence of a monomeric gas. The method of insertion can be through a cut-down or percutaneously (Seldinger Technique).

The heating element's method of heating can be by an electrical heating, radiofrequency, laser, or small coils through which hot solution can be circulated. The heating element should not exceed about 50° C. at the surface that contacts blood.

Such a heating element can be used for core heating and can also be used for local or regional heating. For example, a percutaneous insertion into an artery with a hollow sheath or catheter can be made to accommodate a steering guidewire so the device can be placed into the hepatic artery. A second hollow catheter with a thermistor tip can be placed, via a venous percutaneous stick, into the hepatic vein for liver temperature.

Methods which heat the blood to raise the core temperature, such as extracorporeal whole body hyperthermia, are preferred. However, methods in which the core temperature is raised by other methods such as by infrared radiation, convection, or surface contact such as a heating blanket can also be used in the method of the invention.

The core temperature can be raised to a temperature range of from about 38 to about 48° C., more preferably about 38 to about 44° C., more preferably about 41.8 to about 42.2° C. The core temperature can be raised for a period of from about 2 minutes to about sixteen hours, a period of from about one-half to about three hours, a period of from about one to about two hours, a period of from about 80 to about 100 minutes, or for about 90 minutes. The core temperature can be taken rectally. For purposes of exemplary temperatures presented in this application, the “core temperature” means rectal temperature. Temperatures other than the rectal temperature can be taken in the practice of the invention, e.g., esphogeal, bladder, tympanic, or cardiac line temperatures. The relationship between such other temperatures and the rectal temperature is well known in the art and such measurement by other methods will allow determination of the core temperature as defined herein.

Recommended exposure times for human patients during extracorporeal whole body hyperthermia given in Table 1 below. Dependant on species, the temperature range can shift upwards by about 2 to about 4° C.

TABLE 1

| Core Temperature (° C.) | Exposure (minutes) |
|---|---|
| 39 | 960 |
| 40 | 480 |
| 41 | 240 |
| 42 | 120 |
| 43 | 60 |
| 44 | 30 |
| 45 | 15 |
| 46 | 8 |
| 47 | 4 |
| 48 | 2 |

The patient's pathogenic load can be determined at least once before the core temperature has been raised at least one time; at least once after the core temperature has been raised and returned to normal at least one time; at least two different times after the core temperature has been raised and returned to normal at least one time, or combinations thereof.

The following protocol design illustrates the hyperthermic treatment. Of course, this illustration does not limit the nature of the treatment when used on a non-human patient.

A. Pre Procedure

After the history, physical examination, and laboratory procedures have been completed, and entry criteria satisfied, the patient is admitted to the treatment facility (hospital OR) on the day of the procedure. Blood should be drawn according to the Table of Required Observations. Patient may have Nothing Per Os (NPO) for at least 6 hours prior to the procedure. Preoperative antibiotics are given prophylactically for about 24 hours.

Procedural Parameters

Once in the Operating Room (OR) or treatment room it or the patient is placed on the OR table and prepared for the procedure.

1. Description of Treatment Facility:

The OR or treatment room used for the procedure does not have to be modified for this procedure. The operating table should be equipped with a foam rubber mattress and/or pads for flexor point protection.

2. Patient Instrumentation for Extracorporeal Whole-Body Hyperthermia (herein “EWBH”):

The following should be placed in the operating room prior to EWBH:

i. Swan-Ganz EKG lead monitoring

ii. Peripheral intravenous (IV) lines (2),

iii. Radial artery catheter

iv. Pulmonary artery (Swan-Ganz type) thermistor catheter via central vein.

v. Oximeter.

vi. Urinary bladder catheter with thermistor.

vii. Rectal temperature probe.

viii Esophageal temperature probe (general anesthesia).

ix Tympanic temperature.

x. Bilateral femoral venous catheters is placed by a surgeon and connected to the hyperthermia unit

Temperature probes (esophageal, rectal, and tympanic) should be calibrated, within about 0.1° C., to a NIST traceable device.

3. Anesthesia:

Anesthetic management, if deemed necessary, is the responsibility of the anesthesiologist who must administer the appropriate agents according to the standard of care. The choice of anesthetic agent is determined based on individual patient profile. Either general anesthesia or sedative agents can be used.

To ensure an adequate hourly urine volume, a dopamine drip at about 2 to about 3 mcg/kg/min is used in human patients with adjustments necessary for other patient species based on blood volume, weight and physiological response to high temperature conditions, throughout the procedure and in the early postoperative period. Average urinary flow of about 30-cc/hr minimum is targeted in human patients with species specific adjustments required. Fluid replacement during the procedure is administered at the discretion of the operating team.

4. EWBH conduct, all parameters should be entered on case report forms:

From the Swan Ganz catheter, serial readings of pulmonary systolic and diastolic pressures and blood temperature should be recorded.

Cardiac output (CO) as measured via the thermodilution catheter should be measured prior to and following the treatment.

Each patient should be continuously monitored at 5 minute intervals for temperature during the procedure. The perfusionist should record all perfusion data on specific perfusion data forms. Other patient parameters should be recorded on standard OR flow sheets.

Temperatures Monitored

The following temperatures may be monitored: Rectal (T_{R}), Esophageal (T_{E}), Tympanic (T_{P}), Pulmonary Artery (T_{PA}), Water Inlet/Heat Exchanger (T_{W}), Blood Outlet/Heat Exchanger (T_{Bld})

a. Preparation:

The perfusionist should prime the circuit with an isotonic solution, which should be circulated until totally de-aired. The surgeon should cannulate the femoral veins using open or percutaneous methods for connection with the extracorporeal circuit.

A predetermined dose of heparin required for extracorporeal circulatory bypass should be calculated at 1 50-units/kg and administered in two 75-unit/kg doses with an Activated Clotting Time (ACT) determination before and after each dose. An ACT 2-½ to 3 times normal should be maintained during EWBH. Further doses of heparin, if needed, should be administered according to ACT measurement.

b. Heating Phase:

The time to reach a core temperature of 41.8°±0.2° can take up to approximately 40 minutes.

i. EWBH can be initiated at a blood flow rate of approximately <20% of the baseline cardiac output. The water circulating through the heat exchanger should not exceed 50° C. for longer than 5 minutes.

ii. When either T_{E} or T_{R} (whichever is greater) reaches 41.8°±0.2° C., in human patients, the plateau phase begins.

iii. When about 40.0° C. is reached in human patients, ice packs should be placed under and/or around the patient's neck.

c. Plateau Phase:

i. Core body temperature (T_{E}, or T_{R}, whichever is greater) should be maintained between about 41.6 to about 42.0° C. for approximately 90 minutes. T_{W} should be reduced so that neither T_{E} or T_{R} exceeded about 42.0° C. Since body temperature cannot be instantaneously changed, momentary excursions above 42.0° C. are not to be considered protocol deviations.

ii. Blood flow should be altered to regulate blood and core temperature.

d. Cooling Phase:

In human patients, the anticipated time to reach 39° C. is about 30 to 45 minutes.

i. Cooling should be initiated at first by discontinuing the water flow for about the first 20 minutes, cooling by ambient heat loss.

ii. After about 20 minutes the thermostat should be reset to about 30° C. in human patients, and the water flow re-instituted.

iii. When T_{R} reaches about 39° C., bypass should be discontinued in human patients.

iv. Decannulation and reversed heparin with protamine sulfate.

e. Once stable, the patient should be transferred to the post anesthesia or recovery room.

REQUIRED OBSERVATIONS DURING EWBH BY PHASE

| | | Warming | | | | Plateau | | | | | | | Cooling | | |
|---|---|---|---|---|---|---|---|---|---|---|---|---|---|---|---|
| | Pre/ | 0 | 15 | 30 | 45 | 0 | 15 | 30 | 45 | 60 | 75 | 90 | 15 | 30 | |
| Test* | | | | | | | | | | | | | | | |
| Post Blood Gases | | X | X | X | X | X | X | X | X | X | X | X | X | X | X |
| Electrolytes | X | | | X | | | | X | | | | X | | X | |
| Biochemistry | X | | | | | | | | | | | | | X | |
| Hematology | X | | | | | | | | | | | | | | X |
| Urine Analysis & Culture | X | | | | | | | | | | | | | | |
| CD4/CD8 | X | | | | | | | | | | | | | | |
| Pathogen Specific Assay | X | | | | | | | | | | | | | | |
| Coagulation | X | | | | | | | | | | | | | | X |
| ACT only | X | X | X | X | X | X | X | X | X | X | X | X | X | X | X |
| Cardiac Output | | X | — | — | — | — | — | — | — | — | — | — | — | — | X |
| Urine Output | X— | — | — | — | — | — | — | — | — | — | — | — | X | | |
| Pressure | | | | | | | | | | | | | | | |
| Arterial | X — | — | — | — | — | — | — | — | — | — | — | — | X | | |
| Pulmonary | X — | — | — | — | — | — | — | — | — | — | — | — | X | | |
| EKG | X — | — | — | — | — | — | — | — | — | — | — | — | X | | |
| Temperature | X — | — | — | — | — | — | — | — | — | — | — | — | X | | |

Legend:
X = Discreet sample/monitor point
X—X = Continuous monitoring recorded at 15 ± 5 minute intervals
*Tests may be performed at any time following intervention.

C. Post-EWBH Patient Monitoring

1. In the Post Anesthesia or Recovery Room, standard monitoring should include:

Continuous EKG monitoring,

12 lead EKG strip if indicated,

Temperature, pulse, respirations and blood pressures (every 15 minutes for the first one and one-half hours, then every half hour for the next one and one-half hours), and

Urinary output.

2. At the time of discharge from the treatment facility, a chest X-Ray should be obtained to rule out the presence of pulmonary problems such as pneumothorax, atelectasis, etc. Pressure dressing should be removed from the femoral cannulation sites to confirm hemostasis.

3. Patients can be discharged from the treatment facility when able to ambulate, approximately 23 hours after admission for human patients.

### Follow-up Visits

Follow-up visits are required at day 1 between day 3-7, and 1 month (±7 days), 2 months (±7 days), 4 months (±7 days), and 6 months (±7 days) after the EWBH treatment (to the extent the patient has reached these time points). At follow-up visits the patient should be examined for possible adverse reactions since their last visit, and any reaction recorded on the case report form. Blood should be drawn for clinical laboratory tests according to the Table of Required Observations.

### Equipment Used

The contents of the following U.S. patents and patent applications are hereby incorporated by reference into this application: (1) U.S. Pat. No. 6,415,797, issued Jul. 9, 2002, and entitled “Treatment of human herpesviruses using hyperthermia” (2) U.S. Pat. No. 6,406,452, issued Jun. 18, 2002, and entitled “Bladder catheter for hyperthermia system” (3) U.S. Pat. No. 6,347,633, issued Feb. 19, 2002, and entitled “Treatment of hepatitis C using hyperthermia” (4) U.S. Pat. No. 6,336,911, issued Jan. 8, 2002, and entitled “Thermal sensor for hyperthermia system” (5) U.S. Pat. No. 5,391,142, issued Feb. 21, 1995, and entitled “Apparatus and Method for the Extracorporeal Treatment of the Blood of a Patient Having a Medical Condition,” (6) U.S. Pat. No. 5,674,190, issued Oct. 7, 1997, and entitled “Extracorporeal Whole Body Hyperthermia Using Alpha-Stat Regulation of Blood pH and pCO₂,” (7) U.S. patent application Ser. No. 09/334,224, filed Jun. 16, 1999, entitled “Bladder Catheter for Hyperthermia System,” and (8) U.S. patent application Ser. No. 09/334,520, filed Jun. 16, 1999, entitled “Thermal Sensor for Hyperthermia System.”

The hyperthermia equipment to be used is composed of three main components: (a) the console, (b) a heater/cooler unit and (c) the disposable blood contact circuit.

The console is composed of an extracorporeal, centrifugal pump device used for the operating and monitoring of the hyperthermia procedure. It contains the drive motor and controllers for the pump and electronics for monitoring the system parameters (temperature, pressure, and flow). The heater/cooler unit is used to raise or lower the patient's temperature and maintain a desired patient temperature through conductive heat transfer. Heated water is circulated through the heat exchanger to elevate the patient's temperature. Cool water is circulated through the heat exchanger to reduce the patient's temperature.

The disposable blood contact circuit is comprised of components for inducing and monitoring hyperthermia. In order to complete the circuit, vascular access is required. Blood flows from the patient via a venous cannula and PVC tubing, which is directed to a centrifugal pump. From the pump, the blood is propelled through the heat exchanger where thermal exchange occurs, with the assistance of the heater/cooler. After the blood is heated it passes through a blood filter before returning to the patient via a second venous cannula. A calibrated thermistor probe placed within the outlet of the heat exchanger monitors circuit temperature. This represents the highest blood temperature reading in the circuit. The blood temperature and those temperatures recorded from the heater/cooler as well as patient temperatures are the basis of the perfusion management of blood flow and heater/cooler temperature during the procedure.

Circuit flow is measured by an electrically isolated electromagnetic flowmeter built into the console, and a flow insert that is located in the blood circuit. Flow rates values have been determined experimentally to be approximately <20% of the baseline cardiac output. At these flow levels the rate of temperature rise to the patient is gradual enough not to cause biochemical parameters to change drastically. Blood flow rate adjustment is used with water bath temperature adjustment to fine tune the process and maintain the core body temperature within a narrow range for the appropriate time.

Circuit pressure monitoring is accomplished by the pressure electronics built into the console and a disposable transducer, which is located at the input side of the heat exchanger. This position within the circuit allows the operator to monitor resistance to flow downstream of the pump. Changes in the pressure reading are used as a diagnostic tool to determine circuit integrity and the state of anticoagulation. A connection is made between the three-way stopcock, at the transducer, and the two-way stopcock at the pump input. With the three-way stopcock turned to isolate the pump inlet pressure, the operator is able to recognize a possible malposition of the egress cannula. By utilizing this reading in conjunction with the pulmonary artery diastolic pressure it is possible to anticipate changes in the patient's volume status. A 40 μm filter keeps blood free of particulate matter.

The system is used to perform hyperthermia treatment of the patient's blood. The components and sub-assemblies are consolidated and coordinated to facilitate implementation of use. The apparatus includes structures which define an extracorporeal blood flow circuit. Such a circuit includes a first cannula for use in cannulating a femoral vein of the patient. Such a cannula defines a blood egress point. A second cannula is used for cannulating a different femoral vein of the patient, and the second cannula defines a blood ingress point. A discontinuous conduit is provided to interconnect, in part, the first and second cannulae. A conduit portion of an integrated, sterile module has interposed therein a pump, a heat exchanger for regulating the temperature of blood flowing through the conduit portion, and sensors for ascertaining the temperature, pressure, and flow rate of blood passing through the conduit portion. The apparatus, further, employs a controller for regulating the pump and temperature regulators in response to temperature, pressure, and blood flow rates sensed by the sensors.

A console is employed with the module having various controls. Such controls are used for selectively changing settings to achieve desired pressure and blood flow rate through the conduit portion.

The integrated, sterile module is a disposable component. As a result, the medical treatment facility prevents the possibility of contaminating the blood of one patient with blood of a patient previously treated, and helps safeguard health care workers involved in the treatment.

In cannulating a patient for extracorporeal blood circulation, a blood flow circuit is defined between a first point of cannulation at a vein of the patient and a second point of cannulation at a vein of the patient. The patient's blood is then pumped through the circuit. As the blood passes through the circuit, it is heated to a first elevated temperature for a relatively short period of time. Thereafter, it is heated to a second elevated temperature, lower than the first elevated temperature, for a more extended period of time.

In one embodiment of the invention, the blood is heated to a first elevated temperature of between about 42° C. to about 48° C. Dependent on species, the blood can, typically, be maintained at the first elevated temperature for a period of time of about one half to one hour. Thereafter, the blood can be maintained at the second elevated temperature for a period of about one to two hours. The second elevated temperature, again species specific, it is envisioned, could be between about 42 to about 44° C. or about 37° C. to about 39° C.

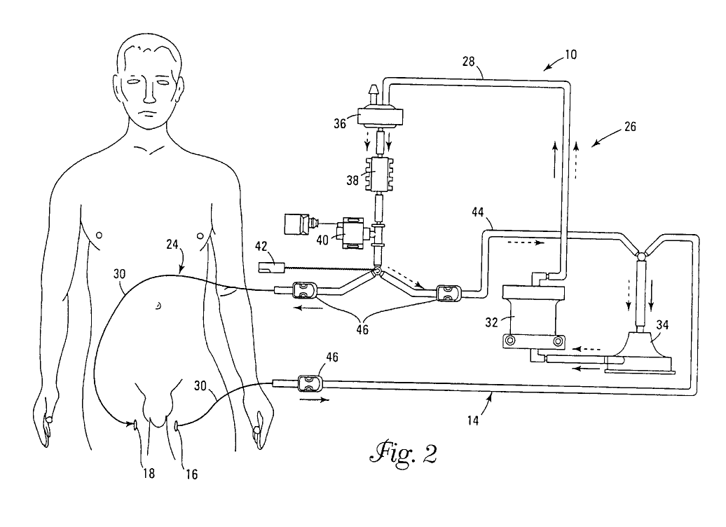

Referring now to the drawings wherein like reference numerals denote like elements through the several views, FIG. 2 shows diagrammatically the apparatus 10 used in the hyperthermia treatment of the human patients as a procedure for addressing biological warfare agent infection. In FIG. 2, a femoral vein in the left leg is cannulated as a point of egress of blood from the patient's body (as at 16), and a femoral vein in the patient's right leg is cannulated as a point of ingress of the blood back into the patient (as at 18). It will be understood that these two specific points of cannulation 16, 18 are not exclusive and that other cannulation locations can be specifically contemplated for human patients. The locations illustrated in FIG. 2, however, have been found to be particularly appropriate in human patients, and ingress and egress points in different legs have been shown as being utilized so that a single leg of the patient is not compromised.

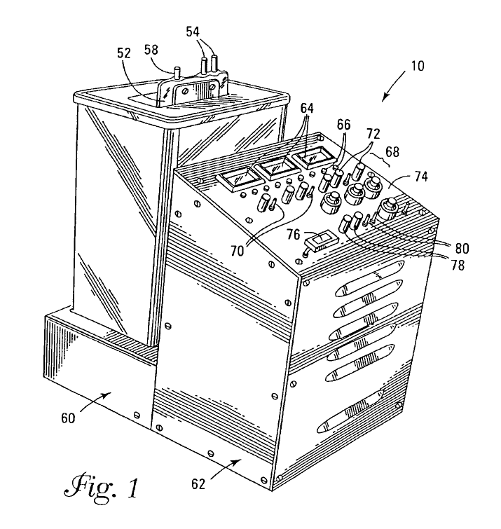

FIG. 2 illustrates the series blood flow circuit 14, which includes first and second cannulae for cannulating the patient at two veins, as previously discussed. A conduit 24 having a discontinuity therein is provided to interconnect, in part, the first and second cannulae. An integrated, sterile module 26, as best seen in FIG. 1, is interfaced with the discontinuity in the discontinuous conduit 24 to complete the series blood flow circuit 14. The module 26 contains all of the components which are exposed to blood in the course of a treatment. It includes a conduit portion 28 which is placed in communication with segments 30 of the discontinuous conduit 24 to complete the circuit 14.

The conduit portion 28 of the disposable module 26 has different components interposed therein. Blood is pumped from the egress point 16 of cannulation at a vein to a heat exchanger 32 by means of a pump 34 of appropriate construction. FIG. 2 illustrates the centrifugal pump 34 that can be used, but it will be understood that this specific type of pump is not exclusive.

FIG. 2 illustrates a heat exchanger 32 down-flow from the pump 34. The heat exchanger 32, functions to selectively elevate the temperature of the blood to a desired level. The blood, after passing through the heat exchanger 32, is passed through a perfusate filter 36. At this location, the perfusate can be purged of any impurities.

A flow probe or sensor 38 is in the series flow circuit 14 down-flow from the perfusate filter 36. The probe 38 serves to sense information with regard to the measure of flow rate of the perfusate passing through the circuit 14. FIG. 2 illustrates the pressure transducer 40 that is used in the circuit 14 down-flow from the flow sensor 38. While it is important to know the flow rate of the perfusate through the circuit 14, it is also important to know the pressure through the system. Consequently, the patient being treated can be adequately protected.

FIG. 2 also illustrates the temperature sensor 42 that is used in the circuit 14. The sensor 42, of course, serves to provide information with regard to the temperature of the blood flowing through the circuit 14.

FIG. 2 also shows a branch 44 of the circuit 14 which recirculates excess perfusate, not needed to be fed back into the patient, back to the pump 34 for recirculation. The recirculation branch 44 is also used during initial setup.

Also illustrated are a series of tubing clamps 46. Such clamps 46 serve, basically, as occluders that can be disposed to pinch tubing segments to preclude flow there through. In FIG. 2, the three such tubing clamps 46 that can be used are illustrated. A first can be immediately down-flow of the egress point on the patient. A second can be located immediately prior to the location at which the blood reenters the patient's body. The third can be positioned in the recirculation segment of the circuit 14.

FIG. 1 illustrates, as previously discussed, an integrated, sterile module 26 in which are disposed all of the components described with reference to FIG. 2 as being exposed to blood in the blood flow circuit 14. FIG. 1 also, however, illustrates the non-disposable base unit that is used including a chassis 60 that removably mounts the integrated, sterile module 26. FIG. 1 further shows that the base unit includes a console or controller unit 62 for controlling operation of the hyperthermia procedure being performed. The console 62 functions to regulate and maintain perfusate flow rate, pressure, and temperature at desired levels.

The console 62 has a series of digital display windows 64. Such windows 64 read temperature, pressure, and flow rate and display those parameters for both actual sensed values and inputted alarm range settings. Each display 64 is provided with a series of visual alarms (i.e., LED's 66 ) for signaling when, for example, a desired range within which temperature, flow rate, or pressure, is intended to be maintained, is exceeded. A series of alarm setting controls 68 are also shown as being provided. Each window 64 has corresponding upper and lower range controls and an intermediately positioned toggle switch 70. The toggle switch 70 can be toggled between positions representative of upper and lower range settings. When in an upper range setting, for example, the appropriate dial 72 can be maneuvered to adjust the upper range limit.

Finally, the control panel 74 of the console 62 has a lower row of dials, displays, etc. These components include a timer 76, rate and amplitude controls 78 for additional modes of operation (such as a pulsatile mode), and an electronic filter 80 for filtering aberrant amplitude signals regarding, for example, pressure in the circuit 14, etc.

In the structure illustrated in FIG. 1 and used to treat the patients, it is intended that the heater/cooler (not shown) for providing external fluid to the heat exchanger 32 does not comprise part of the console 62. Heat exchange is implemented in a collateral manner known in the prior art.

While not specifically shown in FIG. 1, the console 62 contains therewithin a motor 82 which interfaces, through a wall, with the perfusate pump 34. This is done by providing the motor 82 with a magnetic rotor. As the motor 82 is driven, the rotor is caused to be rotated also. A magnetic element is provided in the pump 34, and such a magnetic element is interfaced, through the wall, with the magnetic rotor. Driving of the rotor, in turn, translates to the operation of the pump 34 to a desired level.

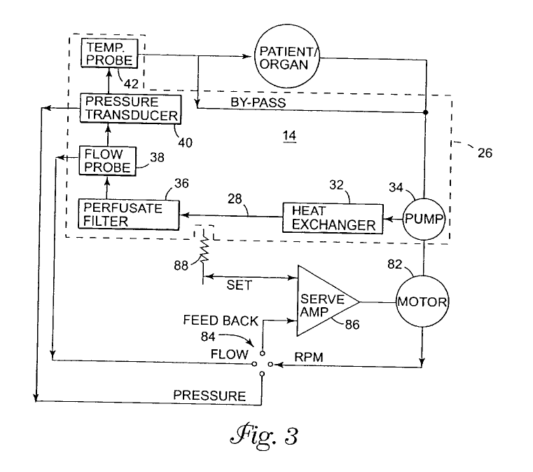

FIG. 3 illustrates schematically how the pump 34, is controlled in response to pressure and flow rate levels sensed by respective sensors 38, 40.

Those figures show the integrated, sterile module 26 and the components enclosed therewithin by a dotted line.

In utilizing the system for hyperthermia treatments, the patient is cannulated in the manner discussed above. Initially, the patient is out of the circuit 14, and flow bypasses the patient. This is effected by manipulation of the appropriate tube clamps 46 to effect flow through the bypass branch circuit 44.

A selector switch 84 is manually positioned so that feedback is provided from either the motor 82, the pressure transducer 40, or the flow probe 38. Input from the appropriate feedback component passes through the selector switch 84 to a servo-amplifier 86. The amplifier 86, in turn, inputs information to control the pump speed in an appropriate fashion to accomplish desired flow and pressure parameters.

FIG. 3 also illustrates a variable resistor 88 which is manipulated in initiating the setting of a particular parameter. The parameter is set and, after the system is appropriately calibrated, the patient is introduced into the flow system 14. Thereafter, continuous monitoring is performed of temperature, pressure, and flow rate. If the alarm system indicates that a parameter has gone outside the desired range, appropriate action can be taken to bring the parameter back within the range.

During hyperthermia, pCO₂ varies directly with a change in body temperature. It is desirable to hold the bloods CO₂ content constant during alpha-stat regulation, thereby requiring an inverse relationship between air convection requirements and body temperature. Alpha-stat maintains constant CO₂ by regulating pCO₂. Hence, utilizing the alpha-stat technique for blood gas management is advantageous in that the pH gradient across the cellular membrane is preserved throughout the range of temperatures encountered during hyperthermia. This alpha-stat regulation of blood pH and pCO₂ is preferred in treating the patients.

By direct control of pulmonary ventilation through manipulation of respiratory rate, the pCO₂, the total CO₂, and the pH are maintained throughout the procedure according to alpha-stat parameters, ensuring that electrolyte balance is maintained throughout. No electrolyte replacement should be required in any patient during the procedure, nor is there ever a need to administer sodium bicarbonate for metabolic acidosis.

The blood flow circuit comprises a Blood Gas Analyzer (BGA). Within the BGA is an analyzer, which analyzes the blood gases, including the blood pH and pCO₂ through infrared or chemical analysis. A pulse oximeter attached to the patient through suitable means, measures the PO₂ of a patient's blood. The microprocessor then analyzes the data associated with the blood's pH, pCO₂, PO₂ and calculates the base excess of the blood normalized at 37° C. The microprocessor is programmed to then automatically adjust the respiratory rate of the patient and either the amount of NaHCO₃ or acidotic crystalloid solution (which affects the HCO₃— ion concentration) being introduced into the patient's blood. This is accomplished by adjusting the respiratory rate of the patient through ventilation or medications.

The respiratory management of the blood at constant CO₂ content, while the temperature changes, maintains a constant alpha thereby stabilizing the biochemical reactions fundamental to the metabolic welfare of components of the patient's blood. In human patients the sodium bicarbonate buffering system is based upon the following equation:

H⁺+HCO₃— ˜H₂CO₃˜H₂O+CO₂

Acidosis (↓pH) occurs when there is an increase of H+ (metabolic) and/or CO₂ (respiratory). Respiratory acidosis is treated with changes in depth of ventilation or ventilatory rate. Metabolic acidosis can be treated with the administration of sodium bicarbonate (NaHCO₃). “Bicarb” dissociates into Na+ and HCO₃— which combines with H+ to form CO₂ and H₂O.

The blood gases, pH, PO₂, pCO₂, and HCO₃— concentration are obtained by direct measurement. Base excess (BE) is a derived parameter based upon the relationship between the measured pCO₂, and HCO₃— concentration, and is calculated relative to the normal HCO₃— centration values: 24 mEq/L in arterial blood and 26 mEq/L in venous blood.

### Optional Equipment

A thermal sensor and bladder catheters that can be used during treatment are described below.

### Thermal Sensor

An improved temperature monitoring device suited to extracorporeal whole body hyperthermia can be used.

The sensor described is connected to the blood flow circuit near the patient. The temperature sensor has a very small mass and is placed on a strut. The strut places the thermal sensor in the laminar blood flow of a duct or fitting. In this fashion, a fast reacting thermal assessment may be made of blood temperature as blood enters or leaves the body.

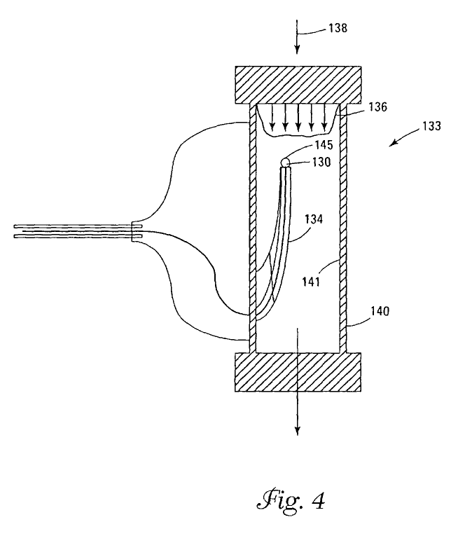

FIG. 4 illustrates a temperature probe 133 for supporting the temperature sensor 130 in the flow of blood moving through a hyperthermia system. As shown in FIG. 4, the probe 133 includes a tube or flow-directing passage 140 having a wall defining an interior lumen 141. Although a cylindrical shape is shown and is preferred to minimize wetted surface area, other cross-sectional shapes are operable. As shown in FIG. 4, the cross-sectional area of the lumen 141 remains constant in the direction of flow indicated by arrow 138. It should be appreciated that the lumen 141 may decrease in cross-sectional area in the direction of flow to maintain laminar flow past the strut 134.

A temperature sensor 130 is attached to the strut 134. Preferably, the strut 134 is shaped and positioned such that the sensor 30 supported thereon is placed in a region of laminar flow and preferably near a location of maximum flow velocity. A region of laminar flow is illustrated in the velocity profile 136. More specifically, the strut 134 is shaped and positioned such that at least a portion of strut 134 lies upstream of the site at which the strut 134 attaches to or passes through the tube 140. The preferred strut 134 has a generally arcuate shape along its length. As shown in the embodiment illustrated in FIG. 4, the strut 134 has a terminating tip 145 that is positioned near the axial center of the tube 140 where the blood flow achieves maximum velocity. In this fashion the sensor 130 is located in the maximum flow zone in the device and can sense subtle changes in blood temperature. By positioning the sensor “in-line,” or in the flow of blood as it passes through the system, advantages are achieved. For example, the laminar flow prevents disruption of the blood and temperature change due to mixing. This factor combined with the fast response small thermal mass sensor 130 improves control of body temperature.

The preferred form of the probe 133 includes fittings which may be barbed. These allow the device to be positioned close to the patient. It is believed that monitoring in close proximity to the patient is desirable to minimize heat loss to the environment.

More than one sensor can be used in a hyperthermic system. The use of a second sensor increases the ability of the system to accurately monitor and control temperature.

The sensors 130 and 132 may be of any temperature-sensing type, such as thermistors, thermocouples, and the like.

### Bladder Catheter

An improved catheter can be used in the whole body hyperthermia system. In use, the catheter is suspended in the bladder of the patient. A cuff on the catheter inflates after the catheter is inserted in the bladder to assist in positioning and securing the catheter. The catheter has a temperature sensor proximal of the inflatable cuff to measure body temperature at the urinary sphincter muscle. The sensor is located relative to the cuff a distance known to generally correspond to the typical distance between the bladder and the sphincter muscle in humans. This distance varies with species and is known to be approximately the same amongst humans regardless of size.

In an alternative catheter, a second temperature sensor is placed distal of the inflatable cuff and thus monitors the temperature of the urine pool in the bladder. Each of the measurements from the first and second temperature sensors has a different time constant depending on the volume of urine in the bladder, and the level of perfusion in the sphincter. Data from these two sensors, the differences between the readings, and the time-dependent variation of these two sensors can contribute to the overall efficacy of the device.

An exemplary version of the bladder catheter is shown in the figures in which like reference numerals refer to equivalent structure throughout.

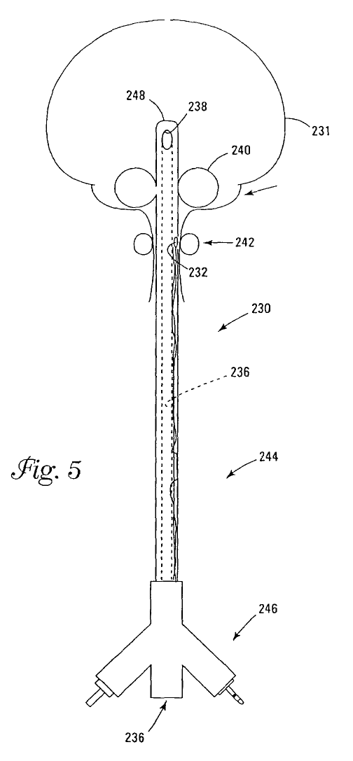

FIG. 5 shows a human bladder temperature probe 230 having an elongate body 244 and terminating in a proximal end 246 and further having a distal tip 248 and a first temperature sensor 232, which may be of any conventional type, including thermistors, thermocouples or other solid state temperature sensors. A drainage lumen 236 communicates with a distal opening 238 to allow fluid to be withdrawn from the bladder 231 or to allow fluid, such as saline, to be infused into the bladder. An inflatable distal cuff 240 positions the catheter and prevents its removal from the bladder while the cuff is inflated. The sensor 232 and the inflatable cuff are spaced and oriented such that when the inflatable cuff 240 holds the probe 230 in position in the patient's bladder 231, the sensor 232 is located proximal of the urinary sphincter muscle 242. Temperature information gathered at this site from the surrounding tissue is likely to be reliable and somewhat less subject to rapid fluctuation than a temperature reading taken from other locations, such as the urine pool.

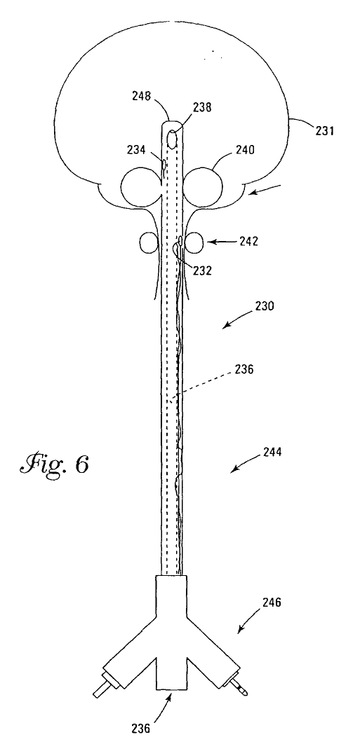

In an alternate catheter for use in humans, illustrated in FIG. 6, the catheter carries a second temperature sensor 234. In practice, the cuff positions the second temperature sensor 234 in the bladder urine or fluid pool while the first sensor 232 is located adjacent the musculature near the sphincter 242. It is expected that the two sensors will vary in measured temperature as the effective time constants for the two locations differ. These two temperatures and relative rates of their variation contribute to the efficacy of body temperature control.

Dimensional and design modifications of the all the apparatus, catheters and sensors can be made to accommodate application to varying species of the animal kingdom.

Computerized controls can be added to all of the equipment described above.

A first preferred embodiment for this invention includes treating a patient infected with any contaminant, where the contaminant is other than a human hepatitis C virus, an HIV virus or a human herpes virus, comprising raising the core temperature of the patient and then returning the core temperature of the patient to normal at least one time, wherein the core temperature of the patient is raised to a temperature range and a duration sufficient to reduce the patient's contaminant level by about 30 percent or more about one month after the core temperature has been raised and returned to normal at least one time.

A further embodiment includes the method of treating a patient as described above, including wherein the patient's contaminant level is determined at least once after the core temperature has been raised and returned to normal at least one time.

A further embodiment includes the method of treating a patient as described above, including wherein the core temperature of the patient is raised and returned to normal two or more times.

A further embodiment of the invention includes the method of treating a patient as described above, including wherein the core temperature is raised by circulating the patient's blood from the patient, through an extracorporeal blood flow circuit, and back to the patient, wherein the blood returned to the patient has been heated within the blood flow circuit to an elevated temperature range.

A further embodiment includes the method of treating a patient as described above, including wherein the core temperature is raised to a temperature range of from about 38° C. to about 44° C.

A further embodiment includes the method of treating a patient as described above, including wherein the core temperature is raised for a period of from about 2 minutes to about sixteen hours.

A further embodiment includes the method of treating a patient as described above, including wherein the core temperature is raised for a period of from about one-half to about three hours.

A further embodiment includes the method of treating a patient as described above, including wherein the patient's contaminant level is determined at least once before the core temperature has been raised at least one time.

A further embodiment includes the method of treating a patient as described above, including wherein the patient's contaminant level is reduced by about 50 percent or more one month after the core temperature has been raised and returned to normal at least one time.

A further embodiment includes the method of treating a patient as described above, including wherein the patient's contaminant level is reduced by about 75 percent or more one month after the core temperature has been raised and returned to normal at least one time.

A further embodiment includes the method of treating a patient as described above, including wherein the patient's contaminant level is reduced by about 90 percent or more one month after the core temperature has been raised and returned to normal at least one time.

A further embodiment includes the method of treating a patient as described above, including wherein the patient's contaminant level is reduced by about 95 percent or more one month after the core temperature has been raised and returned to normal at least one time.

A further embodiment includes the method of treating a patient as described above, including wherein the contaminant level is reduced to less than the sensitivity level of the current state of the art detection method one month after the core temperature has been raised and returned to normal at least one time.

A further embodiment includes the method of treating a patient as described above, including wherein the contaminant is selected from the group consisting of a biological warfare agent, a chemical warfare agent, a benign tumor, a malignant tumor, a mutated cell, a genetically modified biological warfare agent, and any prion, and any combination thereof.

A further embodiment includes the method of treating a patient as described above, including wherein the contaminant has caused an acute, latent, or chronic equine herpes virus infection.

A further embodiment includes the method of treating a patient as described above, including wherein the contaminant has caused an equine encephalitis infection in the patient.

A further embodiment includes the method of treating a patient as described above, including wherein the contaminant has caused a West Nile Virus infection in the patient.

A further embodiment includes the method of treating a patient as described above, including wherein the pharmaceutical or other agent is indicated for a contaminant or used to boost the patient's immune system.

A further embodiment includes the method of treating a patient as described above, including wherein a pharmaceutical or other agent indicated for the contaminant or used to boost the immune system is administered before raising the core temperature of the patient at least one time.

A further embodiment includes the method of treating a patient as described above, including wherein the pharmaceutical or other agent is administered while the core temperature of the patient is raised.

A further embodiment includes the method of treating a patient as described above, including wherein the pharmaceutical or other agent is administered after the core temperature of the patient has been raised and returned to normal at least one time.

A further embodiment includes the method of treating a patient as described above, including wherein the pharmaceutical or other agent is selected from the group consisting of interferons, protease inhibitors, cytokines and chemotherapeutic agents, and combinations of those things.

A further embodiment includes the method of treating a patient as described above, including wherein the pharmaceutical or other agent is selected from the group consisting of ribavirin, lamivudine, alpha interferon, doxorubicin, liposomal doxorubicin, interferon alfacon-1, interferon alfa-2a, interferon alfa-2b, interferon-alfa-n1, thymosin alpha-1, interleukin-2, interferon alpha-n3, ketoprofen, interferon beta-1a, interferon gamma-1b, interleukin-12, histamine dihydrochloride, thymalfasin, zidovudine, didanosine, zalcitabine, stavudine, abacavar, nevirapine, delaviridine, efavirenz, ritonavir, indinavir, nelfinavir, saquinavir, amprenavir, doxorubicim, aciclovir, cidofovir, famciclovir, foscamet, ganciclovir, idoxuridine, trifluorothymidine, valaciclovir, and vidarabine, and combinations of those things.

A further embodiment includes the method of treating a patient as described above, including wherein the contaminant causes an acute infection, a latent infection, or a chronic infection.

A further embodiment includes the method of treating a patient as described above, including wherein the patient is infected with a secondary contaminant.

A further embodiment includes the method of treating a patient as described above, including wherein the secondary contaminant with which the patient is infected is selected from the group consisting of a virus, spirochete, a bacterium, any heat labile virus, any heat labile spirochete, any heat labile bacterium, any heat labile parasite, any heat labile benign or malignant cancer cell, any prion, and any chemical used as a warfare agent.

A further embodiment includes the method of treating a patient as described above, including wherein the secondary contaminant is a spirochete selected from the group consisting of Treponema pallidum, Treponema pertenue, Treponema carateum, Treponema pallidum endemicum, Borrelia burgdorferi, Borrelia hermsii, and Leptospira interrogans.

A further embodiment includes the method of treating a patient as described above, including wherein the secondary contaminant is a spirochete selected from the group consisting of spirochetes of the genus treponema, spirochetes of the genus borrelia, and spirochetes of the genus leptospira.

A further embodiment includes the method of treating a patient as described above, including wherein the contaminant within the patient is an organism.

A further embodiment includes the method of treating a patient as described above, including wherein the contaminant within the patient is selected from the group consisting of any genetically modified virus, any spirochete, any bacterium, any genetically modified spirochete, any genetically modified bacterium, or any virus that is not a Hepatitis C virus, a human herpes virus, or an HIV virus.

A further embodiment includes the method of treating a patient as described above, including wherein the contaminant is a spirochete selected from the group consisting of spirochetes of the genus trepanema, spirochetes of the genus borrelia, and spirochetes of the genus leptospira.

A further embodiment includes the method of treating a patient as described above, including wherein the contaminant is a spirochete selected from the group consisting of Treponema pallidum, Treponema pertenue, Treponema carateum, Treponema pallidum endemicum, Barrelia burgdorferi, Borrelia hermsii and Leptospira interrogans.

A further embodiment includes the method of treating a patient as described above, including wherein the contaminant is any parasite, any genetically modified parasite, any malignant or benign cancer cell, any fungus type, any genetically modified fungus type, any yeast type, or any genetically modified yeast type.

A further embodiment includes the method of treating a patient as described above, including wherein the patient has received any treatment available for a condition arising from the presence of the contaminant but has not responded to that treatment.

A further embodiment includes the method of treating a patient as described above, including wherein the patient has received any treatment available for a condition arising from the presence of the contaminant and has marginally responded to that treatment.

A further embodiment includes the method of treating a patient as described above, including wherein the patient has received any treatment for a condition arising from the presence of the contaminant and has responded to that treatment, but has not been able to resolve the condition.

A further embodiment includes the method of treating a patient as described above, including wherein the contaminant has an identity that is unknown at the time of initiation of treatment.

A second preferred embodiment of the invention includes a method for treating a patient infected with any contaminant, wherein the contaminant is other than a human hepatitis C virus, an HIV virus, or a human herpes virus, comprising raising the core temperature of the patient and then returning the core temperature of the patient to normal at least one time, wherein the core temperature is raised to a temperature range and a duration sufficient to reduce the patient's contaminant level by about 30 percent or more up to three months after the core temperature has been raised and returned to normal at least one time.

A further embodiment includes the method of treating a patient as described above in the second preferred embodiment, including wherein the patient's contaminant level is determined at least once after the core temperature has been raised and returned to normal at least one time.

A further embodiment includes the method of treating a patient as described above in the second preferred embodiment, including wherein the core temperature of the patient is raised and returned to normal two or more times.

A further embodiment of the invention includes the method of treating a patient as described above in the second preferred embodiment, including wherein the core temperature is raised by circulating the patient's blood from the patient, through an extracorporeal blood flow circuit, and back to the patient, wherein the blood returned to the patient has been heated within the blood flow circuit to an elevated temperature range.

A further embodiment includes the method of treating a patient as described above in the second preferred embodiment, including wherein the core temperature is raised to a temperature range of from about 38° C. to about 44° C.

A further embodiment includes the method of treating a patient as described above in the second preferred embodiment, including wherein the core temperature is raised for a period of from about 2 minutes to about sixteen hours.

A further embodiment includes the method of treating a patient as described above in the second preferred embodiment, including wherein the core temperature is raised for a period of from about one-half to about three hours.

A further embodiment includes the method of treating a patient as described above in the second preferred embodiment, including wherein the patient's contaminant level is determined at least once before the core temperature has been raised at least one time.

A further embodiment includes the method of treating a patient as described above in the second preferred embodiment, including wherein the patient's contaminant level is reduced by about 50 percent or more one month after the core temperature has been raised and returned to normal at least one time.

A further embodiment includes the method of treating a patient as described above in the second preferred embodiment, including wherein the patient's contaminant level is reduced by about 75 percent or more one month after the core temperature has been raised and returned to normal at least one time.

A further embodiment includes the method of treating a patient as described above in the second preferred embodiment, including wherein the patient's contaminant level is reduced by about 90 percent or more one month after the core temperature has been raised and returned to normal at least one time.

A further embodiment includes the method of treating a patient as described above in the second preferred embodiment, including wherein the patient's contaminant level is reduced by about 95 percent or more one month after the core temperature has been raised and returned to normal at least one time.

A further embodiment includes the method of treating a patient as described above in the second preferred embodiment, including wherein the contaminant level is reduced to less than the sensitivity level of the current state of the art detection method one month after the core temperature has been raised and returned to normal at least one time.

A further embodiment includes the method of treating a patient as described above in the second preferred embodiment, including wherein the contaminant is selected from the group consisting of a biological warfare agent, a chemical warfare agent, a benign tumor, a malignant tumor, a mutated cell, a genetically modified biological warfare agent, and any prion, and any combination thereof.

A further embodiment includes the method of treating a patient as described above in the second preferred embodiment, including wherein the contaminant has caused an acute, latent, or chronic equine herpes virus infection.

A further embodiment includes the method of treating a patient as described above in the second preferred embodiment, including wherein the contaminant has caused an equine encephalitis infection in the patient.

A further embodiment includes the method of treating a patient as described above in the second preferred embodiment, including wherein the contaminant has caused a West Nile Virus infection in the patient.

A further embodiment includes the method of treating a patient as described above in the second preferred embodiment, including wherein the pharmaceutical or other agent is indicated for a contaminant or used to boost the patient's immune system.

A further embodiment includes the method of treating a patient as described above in the second preferred embodiment, including wherein a pharmaceutical or other agent indicated for the contaminant or used to boost the immune system is administered before raising the core temperature of the patient at least one time.

A further embodiment includes the method of treating a patient as described above in the second preferred embodiment, including wherein the pharmaceutical or other agent is administered while the core temperature of the patient is raised.

A further embodiment includes the method of treating a patient as described above in the second preferred embodiment, including wherein the pharmaceutical or other agent is administered after the core temperature of the patient has been raised and returned to normal at least one time.

A further embodiment includes the method of treating a patient as described above in the second preferred embodiment, including wherein the pharmaceutical or other agent is selected from the group consisting of interferons, protease inhibitors, cytokines and chemotherapeutic agents, and combinations of those things.

A further embodiment includes the method of treating a patient as described above in the second preferred embodiment, including wherein the pharmaceutical or other agent is selected from the group consisting of ribavirin, lamivudine, alpha interferon, doxorubicin, liposomal doxorubicin, interferon alfacon-1, interferon alfa-2a, interferon alfa-2b, interferon-alfa-n1, thymosin alpha-1, interleukin-2, interferon alpha-n3, ketoprofen, interferon beta-1a, interferon gamma-1b, interleukin-12, histamine dihydrochloride, thymalfasin, zidovudine, didanosine, zalcitabine, stavudine, abacavar, nevirapine, delaviridine, efavirenz, ritonavir, indinavir, nelfinavir, saquinavir, amprenavir, doxorubicim, aciclovir, cidofovir, famciclovir, foscamet, ganciclovir, idoxuridine, trifluorothymidine, valaciclovir, and vidarabine, and combinations of those things.

A further embodiment includes the method of treating a patient as described above in the second preferred embodiment, including wherein the contaminant causes an acute infection, a latent infection, or a chronic infection.

A further embodiment includes the method of treating a patient as described above in the second preferred embodiment, including wherein the patient is infected with a secondary contaminant.

A further embodiment includes the method of treating a patient as described above in the second preferred embodiment, including wherein the secondary contaminant with which the patient is infected is selected from the group consisting of a virus, spirochete, a bacterium, any heat labile virus, any heat labile spirochete, any heat labile bacterium, any heat labile parasite, any heat labile benign or malignant cancer cell, any prion, and any chemical used as a warfare agent.

A further embodiment includes the method of treating a patient as described above in the second preferred embodiment, including wherein the secondary contaminant is a spirochete selected from the group consisting of Treponema pallidum, Treponema pertenue, Treponema carateum, Treponema pallidum endemicum, Borrelia burgdorferi, Borrelia hermsii, and Leptospira interrogans.

A further embodiment includes the method of treating a patient as described above in the second preferred embodiment, including wherein the secondary contaminant is a spirochete selected from the group consisting of spirochetes of the genus treponema, spirochetes of the genus borrelia, and spirochetes of the genus leptospira.

A further embodiment includes the method of treating a patient as described above in the second preferred embodiment, including wherein the contaminant within the patient is an organism.

A further embodiment includes the method of treating a patient as described above in the second preferred embodiment, including wherein the contaminant within the patient is selected from the group consisting of any genetically modified virus, any spirochete, any bacterium, any genetically modified spirochete, any genetically modified bacterium, or any virus that is not a Hepatitis C virus, a human herpes virus, or an HIV virus.

A further embodiment includes the method of treating a patient as described above in the second preferred embodiment, including wherein the contaminant is a spirochete selected from the group consisting of spirochetes of the genus trepanema, spirochetes of the genus borrelia, and spirochetes of the genus leptospira.

A further embodiment includes the method of treating a patient as described above in the second preferred embodiment, including wherein the contaminant is a spirochete selected from the group consisting of Treponema pallidum, Treponema pertenue, Treponema carateum, Treponema pallidum endemicum, Barrelia burgdorferi, Borrelia hermsii and Leptospira interrogans.

A further embodiment includes the method of treating a patient as described above in the second preferred embodiment, including wherein the contaminant is any parasite, any genetically modified parasite, any malignant or benign cancer cell, any fungus type, any genetically modified fungus type, any yeast type, or any genetically modified yeast type.

A further embodiment includes the method of treating a patient as described above in the second preferred embodiment, including wherein the patient has received any treatment available for a condition arising from the presence of the contaminant but has not responded to that treatment.

A further embodiment includes the method of treating a patient as described above in the second preferred embodiment, including wherein the patient has received any treatment available for a condition arising from the presence of the contaminant and has marginally responded to that treatment.

A further embodiment includes the method of treating a patient as described above in the second preferred embodiment, including wherein the patient has received any treatment for a condition arising from the presence of the contaminant and has responded to that treatment, but has not been able to resolve the condition.

A further embodiment includes the method of treating a patient as described above in the second preferred embodiment, including wherein the contaminant has an identity that is unknown at the time of initiation of treatment.

Another preferred embodiment of the invention includes treating a patient infected with any contaminant wherein the contaminant is other than the human hepatitis C virus, the HIV virus or the human herpes virus, by raising the core temperature of the patient and then returning the core temperature of the patient to normal at least one time, where the core temperature is raised to a temperature range and a duration sufficient to reduce the patient's contaminant level, and then the core temperature is returned to normal at least one time.

The above description is provided for the purpose of describing embodiments of the invention and is not intended to limit the scope of the invention in any way. It will be apparent to those skilled in the art that various modifications and variations can be made without departing from the spirit or scope of the invention. Thus, it is intended that the present invention cover the modifications and variations of this invention provided they come within the scope of the appended claims and their equivalents.

What is claimed is:

1. A method for treating a patient infected with any contaminant wherein the contaminant is other than a human hepatitis C virus, an HIV virus or a human herpes virus, comprising raising the core temperature of the patient and then returning the core temperature of the patient to normal at least one time, wherein the core temperature is raised to a temperature range and a duration sufficient to reduce the patient's contaminant level by about 30 percent or more about one month after the core temperature has been raised and returned to normal said at least one time.

2. The method of claim 1, including wherein the patient's contaminant level is determined at least once after the core temperature has been raised and returned to normal said at least one time.

3. The method of claim 1, including wherein the core temperature of the patient is raised and returned to normal two or more times.

4. The method of claim 1, including wherein the core temperature is raised by circulating the patient's blood from the patient, through an extracorporeal blood flow circuit, and back to the patient, wherein the blood returned to the patient has been heated within the blood flow circuit to an elevated temperature range.

5. The method of claim 1, including wherein the core temperature is raised to a temperature range of from about 38° C. to about 44° C.

6. The method of claim 5, including wherein the core temperature is raised for a period of from about 2 minutes to about sixteen hours.

7. The method of claim 5, including wherein the core temperature is raised for a period of from about one-half to about three hours.

8. The method of claim 1, including wherein the patient's contaminant level is determined at least once before the core temperature has been raised said at least one time.

9. The method of claim 1, including wherein the patient's contaminant level is reduced by about 50 percent or more one month after the core temperature has been raised and returned to normal said at least one time.

10. The method of claim 1, including wherein the patient's contaminant level is reduced by about 75 percent or more one month after the core temperature has been raised and returned to normal said at least one time.

11. The method of claim 1, including wherein the patient's contaminant level is reduced by about 90 percent or more one month after the core temperature has been raised and returned to normal said at least one time.

12. The method of claim 1, including wherein the patient's contaminant level is reduced by about 95 percent or more one month after the core temperature has been raised and returned to normal said at least one time.

13. The method of claim 1, including wherein the contaminant level is reduced to less than the sensitivity level of the current state of the art detection method one month after the core temperature has been raised and returned to normal said at least one time.

14. The method of claim 1, including wherein said contaminant is selected from the group consisting of a biological warfare agent, a chemical warfare agent, a benign tumor, a malignant tumor, a mutated cell, a genetically modified biological warfare agent, and any prion, and any combination thereof.

15. The method of claim 1, including wherein said contaminant has caused an acute, latent, or chronic equine herpes virus infection.

16. The method of claim 1, including wherein said contaminant has caused an equine encephalitis infection in the patient.

17. The method of claim 1, including wherein said contaminant has caused a West Nile Virus infection in the patient.

18. The method of claim 1, further comprising treating the patient with a pharmaceutical or other agent wherein said pharmaceutical or other agent is indicated for a contaminant or used to boost the patient's immune system.

19. The method of claim 18, including wherein said pharmaceutical or other agent is administered before raising the core temperature of the patient said at least one time.

20. The method of claim 18, including wherein said pharmaceutical or other agent is administered while the core temperature of the patient is raised.

21. The method of claim 18, including wherein said pharmaceutical or other agent is administered after the core temperature of the patient has been raised and returned to normal said at least one time.

22. The method of claim 18, including wherein said pharmaceutical or other agent is selected from the group consisting of interferons, protease inhibitors, cytokines and chemotherapeutic agents, and combinations thereof.

23. The method of claim 18, including wherein said pharmaceutical or other agent is selected from the group consisting of ribavirin, lamivudine, alpha interferon, doxorubicin, liposomal doxorubicin, interferon alfacon-1, interferon alfa-2a, interferon alfa-2b, interferon-alfa-n1, thymosin alpha-1, interleukin-2, interferon alpha-n3, ketoprofen, interferon beta-1a, interferon gamma-1b, interleukin-12, histamine dihydrochloride, thymalfasin, zidovudine, didanosine, zalcitabine, stavudine, abacavar, nevirapine, delaviridine, efavirenz, ritonavir, indinavir, nelfinavir, saquinavir, amprenavir, doxorubicim, aciclovir, cidofovir, famciclovir, foscamet, ganciclovir, idoxuridine, trifluorothymidine, valaciclovir, and vidarabine, and combinations thereof.

24. The method of claim 1, including wherein said contaminant causes an acute infection, a latent infection, or a chronic infection.

25. The method of claim 1, including wherein the patient is infected with a secondary contaminant.

26. The method of claim 25, including wherein said secondary contaminant is selected from the group consisting of a virus, spirochete, a bacterium, any heat labile virus, any heat labile spirochete, any heat labile bacterium, any heat labile parasite, any heat labile benign or malignant cancer cell, any prion, and any chemical used as a warfare agent.

27. The method of claim 25, including wherein said secondary contaminant is a spirochete selected from the group consisting of Treponema pallidum, Treponema pertenue, Treponema carateum, Treponema pallidum endemicum, Borrelia burgdorferi, Borrelia hermsii, and Leptospira interrogans.

28. The method of claim 25, including wherein said secondary contaminant is a spirochete selected from the group consisting of spirochetes of the genus treponema, spirochetes of the genus borrelia, and spirochetes of the genus leptospira.

29. The method of claim 1, including wherein said contaminant is an organism.

30. The method of claim 29, including wherein said contaminant is selected from the group consisting of any genetically modified virus, any spirochete, any bacterium, any genetically modified spirochete, any genetically modified bacterium, or any virus that is not a Hepatitis C virus, a human herpes virus, or an HIV virus.

31. The method of claim 29, including wherein said contaminant is a spirochete selected from the group consisting of spirochetes of the genus trepanema, spirochetes of the genus borrelia, and spirochetes of the genus leptospira.

32. The method of claim 29, including wherein said contaminant is a spirochete selected from the group consisting of Treponema pallidum, Treponema pertenue, Treponema carateum, Treponema pallidum endemicum, Barrelia burgdorferi, Borrelia hermsii and Leptospira interrogans.

33. The method of claim 29, including wherein said contaminant is any parasite, any genetically modified parasite, any malignant or benign cancer cell, any fungus type, any genetically modified fungus type, any yeast type, or any genetically modified yeast type.

34. The method of claim 1, including wherein the patient has received any treatment available for a condition arising from the presence of said contaminant and has not responded to said treatment.

35. The method of claim 1, including wherein the patient has received any treatment available for a condition arising from the presence of said contaminant and has marginally responded to said treatment.

36. The method of claim 1, including wherein the patient has received any treatment for a condition arising from the presence of said contaminant and has responded to said treatment, but has not been able to resolve said condition.

37. The method of claim 1, including wherein said contaminant has an identity that is unknown at the time of initiation of treatment.

38. A method for treating a patient infected with any contaminant wherein the contaminant is other than a human hepatitis C virus, an HIV virus, or a human herpes virus, comprising raising the core temperature of the patient and then returning the core temperature of the patient to normal at least one time, wherein the core temperature is raised to a temperature range and a duration sufficient to reduce the patient's contaminant level by about 30 percent or more up to three months after the core temperature has been raised and returned to normal said at least one time.

39. The method of claim 38, including wherein the patient's contaminant level is determined at least once after the core temperature has been raised and returned to normal said at least one time.

40. The method of claim 38, including wherein the core temperature of the patient is raised and returned to normal two or more times.

41. The method of claim 38, including wherein the core temperature is raised by circulating the patient's blood from the patient, through an extracorporeal blood flow circuit, and back to the patient, wherein the blood returned to the patient has been heated within the blood flow circuit to an elevated temperature range.

42. The method of claim 38, including wherein the core temperature is raised to a temperature range of from about 38° C. to about 44° C.

43. The method of claim 42, including wherein the core temperature is raised for a period of from about 2 minutes to about sixteen hours.

44. The method of claim 42, including wherein the core temperature is raised for a period of from about one-half to about three hours.

45. The method of claim 38, including wherein the patient's contaminant level is determined at least once before the core temperature has been raised said at least one time.

46. The method of claim 38, including wherein the patient's contaminant level is reduced by about 50 percent or more up to three months after the core temperature has been raised and returned to normal said at least one time.

47. The method of claim 38, including wherein the patient's contaminant level is reduced by about 75 percent or more up to three months after the core temperature has been raised and returned to normal said at least one time.

48. The method of claim 38, including wherein the patient's contaminant level is reduced by about 90 percent or more up to three months after the core temperature has been raised and returned to normal said at least one time.

49. The method of claim 38, including wherein the patient's contaminant level is reduced by about 95 percent or more up to three months after the core temperature has been raised and returned to normal said at least one time.

50. The method of claim 38, including wherein the contaminant level is reduced to less than the sensitivity level of the current state of the art detection method up to three months after the core temperature has been raised and returned to normal said at least one time.

51. The method of claim 38, including wherein said contaminant is selected from the group consisting of a biological warfare agent, a chemical warfare agent, a benign tumor, a malignant tumor, a mutated cell, a genetically modified biological warfare agent, and any prion, and any combination thereof.

52. The method of claim 38, including wherein said contaminant has caused an acute, latent, or chronic equine herpes virus infection.

53. The method of claim 38, including wherein said contaminant has caused an equine encephalitis infection in the patient.

54. The method of claim 38, including wherein said contaminant has caused a West Nile Virus infection in the patient.

55. The method of claim 38, further comprising treating the patient with a pharmaceutical or other agent wherein said pharmaceutical or other agent is indicated for a contaminant or used to boost the patient's immune system.

56. The method of claim 55, including wherein said pharmaceutical or other agent is administered before raising the core temperature of the patient said at least one time.

57. The method of claim 55, including wherein said pharmaceutical or other agent is administered while the core temperature of the patient is raised.

58. The method of claim 55, including wherein said pharmaceutical or other agent is administered after the core temperature of the patient has been raised and returned to normal said at least one time.

59. The method of claim 55, including wherein said pharmaceutical or other agent is selected from the group consisting of interferons, protease inhibitors, cytokines and chemotherapeutic agents, and combinations thereof.

60. The method of claim 55, including wherein said pharmaceutical or other agent is selected from the group consisting of ribavirin, lamivudine, alpha interferon, doxorubicin, liposomal doxorubicin, interferon alfacon-1, interferon alfa-2a, interferon alfa-2b, interferon-alfa-n1, thymosin alpha-1, interleukin-2, interferon alpha-n3, ketoprofen, interferon beta-1a, interferon gamma-1b, interleukin-12, histamine dihydrochloride, thymalfasin, zidovudine, didanosine, zalcitabine, stavudine, abacavar, nevirapine, delaviridine, efavirenz, ritonavir, indinavir, nelfinavir, saquinavir, amprenavir, doxorubicim, aciclovir, cidofovir, famciclovir, foscamet, ganciclovir, idoxuridine, trifluorothymidine, valaciclovir, and vidarabine, and combinations thereof.

61. The method of claim 38, including wherein said contaminant causes an acute infection, a latent infection, or a chronic infection.

62. The method of claim 38, including wherein the patient is infected with a secondary contaminant.

63. The method of claim 62, including wherein said secondary contaminant selected from the group consisting of a virus, spirochete, bacterium, any heat labile virus, any heat labile spirochete, any heat labile bacterium, any heat labile parasite, any heat labile benign or malignant cancer cell, any prion, and any chemical used as a warfare agent.

64. The method of claim 62, including wherein said secondary contaminant is a spirochete selected from the group consisting of Treponema pallidum, Treponema pertenue, Treponema carateum, Treponema pallidum endemicum, Borrelia burgdorferi, Borrelia hermsii, and Leptospira interrogans.

65. The method of claim 62, including wherein said secondary contaminant is a spirochete selected from the group consisting of spirochetes of the genus treponema, spirochetes of the genus borrelia, and spirochetes of the genus leptospira.

66. The method of claim 38, including wherein said contaminant is an organism.

67. The method of claim 66, including wherein said contaminant is any genetically modified virus, any spirochete, any bacterium, any genetically modified spirochete, any genetically modified bacterium, or any virus that is not the human Hepatitis C virus, the HIV virus, or the human herpes virus.

68. The method of claim 66, including wherein said contaminant is a spirochete selected from the group consisting of spirochetes of the genus treponema, spirochetes of the genus borrelia, and spirochetes of the genus leptospira.

69. The method of claim 66, including wherein said contaminant is a spirochete selected from the group consisting of Treponema pallidum, Treponema pertenue, Treponema carateum, Treponema pallidum endemicum, Borrelia burgdorferi, Borrelia hermsii and Leptospira interrogans.

70. The method of claim 61, including wherein said contaminant is any parasite, any genetically modified parasite, any malignant or benign cancer cell, any fungus type, any genetically modified fungus type, any yeast type, or any genetically modified yeast type.

71. The method of claim 38, including wherein the patient has received any treatment available for a condition arising from the presence of said contaminant and has not responded to said treatment.

72. The method of claim 38, including wherein the patient has received any treatment available for a condition arising from the presence of said contaminant and has marginally responded to said treatment.

73. The method of claim 38, including wherein the patient has received any treatment for a condition arising from the presence of said contaminant and has responded to said treatment, but has not been able to resolve said condition.

74. The method of claim 38, including wherein said contaminant has an identity that is unknown at the beginning of treatment.

75. A method for treating a patient infected with any contaminant wherein the contaminant is other than the human hepatitis C virus, the HIV virus or the human herpes virus, comprising raising the core temperature of the patient and then returning the core temperature of the patient to normal at least one time, wherein the core temperature is raised to a temperature range and a duration sufficient to reduce the patient's contaminant level then returned to normal at least one time.
